(19) United States
(12) Patent Application Publication
(10) Pub. No.: US 2026/0256655 A1
(43) Pub. Date: Sep. 3, 2026

(54) GUIDE FOR MEDICAL INSERTION TOOL

(71) Applicant: JMS Co., Ltd., Hiroshima (JP)

(72) Inventors: Yuma HAYASHI, Hiroshima (JP); Asami UEDA, Hiroshima (JP); Miki ISHIDA, Hiroshima (JP); Nozomi HAMAMURA, Hiroshima (JP); Hiroka SASAKI, Hiroshima (JP); Shinya HATTORI, Hiroshima (JP)

(21) Appl. No.: 18/878,427

(22) PCT Filed: Jun. 27, 2023
(86) PCT No.: PCT/JP2023/023817

§ 371 Date: Dec. 23, 2024

(30) Foreign Application Priority Data
Jun. 28, 2022 (JP) 2022-103990

(51) Int. Cl. A61J 15/00; A61M 25/00; A61M 25/01

(52) U.S. Cl. CPC A61J 15/0003; A61M 25/0023; A61M 25/0054; A61M 25/01

(57) ABSTRACT

An aspect of the present invention relates to a medical-insert guide (1) including a cylindrical component (2). The cylindrical component (2) is configured to be indwelled in a body from a nose or a mouth to an inside of a gastrointestinal tract, and has an inner cavity into which a medical insert can be removably inserted. A portion (23) of the cylindrical component (2) positioned at least at a pharynx and an upper esophageal sphincter when the cylindrical component is indwelled in the body, and an insertion-side front end portion (24) of the cylindrical component (2) each have flexibility that allows the cross-sectional shape of the portion to be deformed into a flat shape by pharynx pressure or esophageal pressure when the cylindrical component is indwelled in the body, or each preliminarily have a flat cross-sectional shape, and each have kink resistance. Alternatively, across the whole length of the cylindrical component (2) in its longitudinal direction, the cylindrical component (2) has flexibility that allows its cross-sectional shape to be deformed into a flat shape by pharynx pressure or esophageal pressure when the cylindrical component is indwelled in the body, or preliminarily has a flat cross-sectional shape, and has kink resistance. It is preferable that the cylindrical component (2) has at least one high-kink-resistance region (2a), and the high-kink-resistance region (2a) extends across the whole length of the cylindrical component in the longitudinal direction, and is less likely to be subjected to compression deformation in the longitudinal direction than a region of the cylindrical component that is adjacent to the high-kink resistance region in the circumferential direction of the cylindrical component is likely to be subjected thereto.

### TECHNICAL FIELD

The present invention relates to, e.g., a medical-insert guide and a medical device for nasal tube feeding that includes the medical-insert guide.

### BACKGROUND ART

Malnutrition of the elderly hospitalized in the acute phase has been a social issue. It has been reported that approximately 40% of the hospitalized elderly suffer from malnutrition. The malnutrition causes slow recovery after surgery, and significantly influences medical expenses and nursing care expenses, for example.

Nasal tube feeding that administers nutrition to the stomach through a tube inserted from the nose has an advantage that it can reliably and efficiently supply necessary nutrition to the stomach of the patient. In the nasal tube feeding, however, a nasal feeding catheter for feeding nutrition to the stomach through the nostril and the esophagus continues being indwelled in a body not only when nutrition administration is performed but also when it is not performed. The catheter has a diameter necessary for feeding nutrition, and a hardness that maintains the shape of its inner cavity when the catheter is pressed by, e.g., the throat or the esophagus. The catheter also has rigidity to the degree that prevents, e.g., buckling of the catheter when it is inserted into a body. Thus, if such a catheter continues being indwelled in the throat and the like, the patient suffers from much pain. Some patients cannot endure the pain and may remove the catheter by themselves.

To solve this problem, for example, Patent Document 1 has proposed an inner-tube-including gastrointestinal tract tube including an outer tube that has a very thin thickness, and an inner tube installed in the outer tube. The inner-tube-including gastrointestinal tract tube is inserted into a patient with the inner tube installed in the outer tube. The inner tube is removed from the outer tube other than when medicament administration or tube nutrition administration is performed to the patient, and only the thin outer tube is indwelled in the patient. Only the outer tube is indwelled in the patient for a long time other than during the tube nutrition administration. When the tube nutrition administration is not performed, the portion of the outer tube that sticks out of the body from the nose is, for example, bent, tied, or twisted, and stuck to the face of the patient with a piece of tape. When the tube nutrition administration is performed, the inner tube is inserted into the outer tube again, and the nutrition is administered through the inner tube.

### PRIOR ART DOCUMENT

### Patent Document

Patent Document 1: JP H11(1999)-244358 A

### DISCLOSURE OF INVENTION

### Problem to be Solved by the Invention

The outer tube in Patent Document 1 is very thin to the degree that allows the outer tube to be, for example, bent, tied, or twisted. Thus, the front end may retreat with respect to the insertion direction by peristalsis of the esophagus or friction against the outer circumference of a catheter inserted into or removed from the outer tube, and may easily move from a desired position. If the outer tube with its front end out of the desired position is used as a guide for insertion of the inner tube (catheter) into the body, the catheter may be wrongly inserted into the trachea, and cause serious complications.

Therefore, an aspect of the present invention relates to a medical-inert guide that allows insertion and removal of a medical insert such as a catheter, and is to be indwelled in a body from the nose or the mouth to the inside of the gastrointestinal tract, and provides a medical-insert guide that suppresses misalignment of the front end of the indwelled medical-insert guide without increasing uncomfortableness during the indwelling, and allows safe insertion and removal of a medical insert.

### Means for Solving Problem

An aspect of the present invention relates to a medical-insert guide including a cylindrical component.

The cylindrical component is configured to be indwelled in a body from a nose or a mouth to an inside of a gastrointestinal tract, and has an inner cavity into which a medical insert can be removably inserted.

A portion of the cylindrical component positioned at least at a pharynx and an upper esophageal sphincter when the cylindrical component is indwelled in the body, and an insertion-side front end portion of the cylindrical component each have flexibility that allows the cross-sectional shape of the portion to be deformed into a flat shape by pharynx pressure or esophageal pressure when the cylindrical component is indwelled in the body, or each preliminarily have a flat cross-sectional shape, and each have kink resistance.

Alternatively, across the whole length of the cylindrical component in its longitudinal direction, the cylindrical component has flexibility that allows its cross-sectional shape to be deformed into a flat shape by pharynx pressure or esophageal pressure when the cylindrical component is indwelled in the body, or preliminarily has a flat cross-sectional shape, and has kink resistance.

An aspect of the present invention relates to a medical device for nasal tube feeding including the medical-insert guide according to an aspect of the present invention, and a medical insert. The medical insert is a feeding catheter.

### Effects of the Invention

According to an aspect of the present invention, the medical-insert guide includes the cylindrical component. The portion of the cylindrical component positioned at least at a pharynx and an upper esophageal sphincter when the cylindrical component is indwelled in the body, and the insertion-side front end portion of the cylindrical component each have flexibility that allows the cross-sectional shape of the portion to be deformed into a flat shape by pharynx pressure or esophageal pressure when the cylindrical component is indwelled in the body, or each preliminarily have a flat cross-sectional shape, and each have kink resistance.

Alternatively, across the whole length of the cylindrical component in its longitudinal direction, the cylindrical component has flexibility that allows its cross-sectional shape to be deformed into a flat shape by pharynx pressure or esophageal pressure when the cylindrical component is indwelled in the body, or preliminarily has a flat cross-sectional shape, and has kink resistance. Thus, misalignment of the front end of the indwelled cylindrical component is suppressed without increasing uncomfortableness during the indwelling.

Therefore, the medical insert guide of an aspect of the present invention can achieve both the decrease in uncomfortableness during the indwelling and safe insertion and removal of a medical insert.

### BRIEF DESCRIPTION OF DRAWINGS

FIG. 1 is a perspective view of a medical-insert guide of an aspect of the present invention.

FIG. 2 FIG. 2A is an enlarged view of the base-side end portion of the medical-insert guide illustrated in FIG. 1. FIG. 2B is a cross-sectional view of the medical-insert guide.

FIG. 3 is a cross-sectional view of the medical-insert guide illustrated in FIG. 1 pressed in its radial direction.

FIG. 4 is a cross-sectional view of a medical-insert guide of another aspect of the present invention.

FIG. 5 FIG. 5A is an enlarged view of the base-side end portion of a medical-insert guide of another aspect of the present invention. FIG. 5B is a cross-sectional view of the medical-insert guide.

FIG. 6 is a cross-sectional view of a medical-insert guide of another aspect of the present invention.

FIG. 7 FIG. 7A is an enlarged view of the base-side end portion of a medical-insert guide of another aspect of the present invention. FIG. 7B is a cross-sectional view of the medical-insert guide.

FIG. 8 is a cross-sectional view of the medical-insert guide illustrated in FIG. 7 pressed in its radial direction.

FIG. 9 FIG. 9A is an enlarged view of the base-side end portion of a medical-insert guide of another aspect of the present invention. FIG. 9B is a cross-sectional view of the medical-insert guide.

FIG. 10 is a cross-sectional view of the medical-insert guide illustrated in FIG. 9 pressed in its radial direction.

FIG. 11 FIG. 11A is an enlarged view of the base-side end portion of a medical-insert guide of another aspect of the present invention. FIG. 11B is a cross-sectional view of the medical-insert guide.

FIG. 12 is a cross-sectional view of a medical-insert guide of another aspect of the present invention.

FIG. 13 FIG. 13A is an enlarged view of the base-side end portion of a medical-insert guide of another aspect of the present invention. FIG. 13B is a cross-sectional view of the medical-insert guide.

FIG. 14 is a perspective view of a medical-insert guide of an aspect of the present invention with a camera including shaft inserted therein.

FIG. 15 FIG. 15A is an illustration for explaining an indwelled state of a medical-insert guide of an aspect of the present invention. FIG. 15B is an illustration for explaining a method for sticking the medical-insert guide.

FIG. 16 FIG. 16A is a perspective view of a medical device for nasal tube feeding of an aspect of the present invention. FIG. 16B is a cross-sectional view of the medical device for nasal tube feeding.

FIG. 17 is a drawing that illustrates a state where nasal tube feeding is being performed with the medical device for nasal tube feeding illustrated in FIG. 16.

FIG. 18 is a schematic view for explaining a method for evaluating kink resistance performance.

FIG. 19 FIG. 19A is a schematic view for explaining a state immediately before measurement of bending strength of the insertion-side front end portion of a cylindrical component. FIG. 19B is a schematic view for explaining a state where the insertion-side front end portion of the cylindrical component buckles when the bending strength is performed.

FIG. 20 FIG. 20A is a schematic view for explaining a method for evaluating compressibility of a cylindrical component. FIG. 20B is a cross-sectional view of the cylindrical component pressed in its radial direction.

### DESCRIPTION OF THE INVENTION

The above medical-insert guide of an aspect of the present invention includes a cylindrical component to be indwelled in a body. The cylindrical component is to be indwelled in a body of a patient from the nose or mouth to, e.g., the inside of the esophagus, stomach, duodenum, or jejunum. The medical insert may be a camera-including shaft, a guidewire, or a feeding catheter for nasal tube nutrition feeding to the inside of a body.

The medical-insert guide of an aspect of the present invention is to be indwelled in a body, and thus is desirably less uncomfortable during the indwelling. One cause of the uncomfortableness is the cylindrical component pressing the gastrointestinal tract. The uncomfortableness during the indwelling can be reduced if the cylindrical component has flexibility that allows it to be easily deformed into a flat shape by, e.g., pharynx pressure or esophageal pressure, or the cylindrical component preliminarily has a flat shape.

More specifically, in one aspect, a portion of the cylindrical component positioned at least at the pharynx and the upper esophageal sphincter when the cylindrical component is indwelled in a body, and an insertion-side front end portion of the cylindrical component each have flexibility that allows the cross-sectional shape of the portion to be deformed into a flat shape by pharynx pressure or esophageal pressure, or each preliminarily have a flat cross-sectional shape. In another aspect, across the whole length of the cylindrical component in its longitudinal direction, the cylindrical component has flexibility that allows its cross-sectional shape to be deformed into a flat shape by pharynx pressure or esophageal pressure when the cylindrical component is indwelled in the body, or preliminarily has a flat cross-sectional shape.

In an aspect of the present invention, the “being deformed into a flat shape” may indicate that the pressing decreases the height (the length of the cylindrical component in the pressing direction) and increases the width (the length perpendicular to the pressing direction). For example, if the cylindrical component not being pressed has a circular cross-sectional shape, the pressing may change the cross-sectional shape into an elliptical or cocoon-like shape, or into a flat shape to the degree that the inner cavity of the cylindrical component is almost eliminated. Alternatively, if the cylindrical component not being pressed has a flat cross-sectional shape, the pressing may make the cross-sectional shape flatter. The “preliminarily having a flat shape” may indicate that the cylindrical component not being pressed has an elliptical or substantially elliptical cross-sectional shape. Irrespective of the cross-sectional shape of the cylindrical component not being pressed, such as a circular shape or an elliptical shape, it is preferable that the cylindrical component has, e.g., flexibility to the degree that allows the cylindrical component to be easily deformed by being pressed in its radial direction, and prevents the deformed cylindrical component from pressing the internal wall surfaces of, e.g., the pharynx and the esophagus.

The cylindrical component of the medical insert guide of an aspect of the present invention also has kink resistance to suppress misalignment of the front end of the indwelled cylindrical component. More specifically, in one aspect, the portion of the cylindrical component positioned at least at the pharynx and the upper esophageal sphincter when the cylindrical component is indwelled in a body, and the insertion-side front end portion of the cylindrical component each have kink resistance. In another aspect, the cylindrical component has kink resistance across the whole length of the cylindrical component in its longitudinal direction.

Preferably, the cylindrical component has at least one high-kink-resistance region. The high-kink resistance region extends across the whole length of the cylindrical component in the longitudinal direction (which may also be referred to as “axial direction”), and is less likely to be subjected to compression deformation in the longitudinal direction than a region of the cylindrical component that is adjacent to the high-kink resistance region in the circumferential direction (which may also be simply referred to as “adjacent region” below) is likely to be subjected thereto. For example, the high-kink resistance region is linear. While the cylindrical component is indwelled in a body, the adjacent region is more likely to be bent by being pressed by, e.g., pharynx pressure or esophageal pressure than the high-kink resistance region is likely to be bent thereby, and thus the outer shape of the cross section of the cylindrical component deforms into a flat shape. Preferably, the adjacent region easily deforms and has low elasticity (weak force returning to its original shape after bending), and thus has flexibility that prevents the deformed cylindrical component from pressing the inner wall surfaces of, e.g., the pharynx and the esophagus. Such a configuration facilitates control of the kink resistance, easy deformation in the radial direction, and deformation into a flat shape, compared with a cylindrical component having a single kink resistance feature in its entirety. More specifically, for example, the cylindrical component is easily deformed by being pressed in the radial direction, but is less flexible than medical inserts such as catheters. For example, the cylindrical component has a returning force approximately the same as or weaker than a straw made of synthetic resin.

In the medical-insert guide of an aspect of the present invention, the cylindrical component preferably has a plurality of the high-kink resistance regions. The high-kink resistance regions are arranged at predetermined intervals, preferably, at regular intervals along the circumferential direction of the cylindrical component. In this way, if the plurality of high-kink resistance regions are arranged apart in the circumferential direction, the cylindrical component has good molding stability.

In the medical-insert guide of an aspect of the present invention, the cylindrical component preferably has a thick portion where the cylindrical wall of the cylindrical component is relatively thick, and a thin portion where the cylindrical wall is relatively thin. The thick portion is the high-kink-resistance region. Such a configuration in which the high-kink resistance region is provided by controlling the thickness of the cylindrical wall has an advantage that the cylindrical component can be made of one material. In such a configuration, the cylindrical component may have a plurality of the thick portions and a plurality of the thin portions, and the thick portions and the thin portions may be alternately arranged along the circumferential direction of the cylindrical component.

The thickness of the cylindrical component in the thick portion is preferably two times or more and five times or less the thickness of the cylindrical component in the thin portion. Such an aspect is suitable for achieving both the opposite functions of the easy deformation by being pressed by, e.g., pharynx pressure or esophageal pressure and the kink resistance.

The ratio of the thickness of the cylindrical component in the thin portion to the outer diameter of the cylindrical component (the thickness in the thin portion/the outer diameter of the cylindrical component) is preferably 0.10 or less, more preferably 0.08 or less, and further preferably 0.07 or less. Such an aspect is preferable because the medical-insert guide is easily deformed by being pressed by, e.g., pharynx pressure or esophageal pressure. The lower limit of the ratio (the thickness in the thin portion/the outer diameter of the cylindrical component) is preferably 0.01 or more in view of the strength.

In the medical-insert guide of an aspect of the present invention, it is preferable that the cylindrical component has the thick portion by having a protruding row that protrudes toward the inner cavity of the cylindrical component. Such a configuration is advantageous because it reduces the contact area between the medical-insert guide and the medical insert, and thus can reduce the sliding resistance. Because of a similar reason, it is more preferable that the cylindrical component has a plurality of the thick portions and a plurality of the thin portions, and the thick portions and the thin portions are alternately arranged along the circumferential direction of the cylindrical component. Further, because of a similar reason, the surface of the protruding row is preferably a curved surface that is highest in its central portion.

In the medical-insert guide of an aspect of the present invention, the cylindrical component preferably has the thick portion by having a protruding row that protrudes toward the outside of the cylindrical component. Such a configuration reduces the contact area between the cylindrical component and the inside of the body of the patient, and thus can reduce the sliding resistance when the medical-insert guide is indwelled into the body of the patient. In the medical-insert guide of an aspect of the present invention, the cylindrical component may have both the protruding row that protrudes toward the inner cavity of the cylindrical component and the protruding row that protrudes toward the outside of the cylindrical component, and they may constitute the thick portion.

In the medical-insert guide of an aspect of the present invention, it is preferable that the cylindrical component has a plurality of the protruding rows that protrude toward the inner cavity of the cylindrical component, and an imaginary circle that connects the tops of the plurality of protruding rows has a diameter of 1.0 mm or more. Such a configuration is advantageous because it reduces the sliding resistance between the medical-insert guide and the medical insert, and thus allows smooth insertion and removal of feeding catheters of 3 Fr (outer diameter: 1.0 mm), which are often used in nasal tube feeding.

In the medical-insert guide of an aspect of the present invention, it is preferable that the cylindrical component is made of at least two types of materials that differ in hardness, and the hardness of the material of the thick portion is higher than the hardness of the material of the thin portion. Such a configuration is advantageous in the control of the kink resistance, easy deformation in the radial direction, and deformation into a flat shape.

In the medical-insert guide of an aspect of the present invention, it is preferable that the cylindrical wall of the cylindrical component has a region B and a region A that is adjacent to the region B and is made of a material having a higher hardness than the hardness of the material of the region B. The region A is the high-kink-resistance region. The region B and the region A are adjacent to each other along the circumferential direction of the cylindrical component. Such a configuration in which the high-kink resistance region is provided by using a material having a relatively high hardness allows, e.g., the constant thickness of the cylindrical wall of the cylindrical component in the circumferential direction. In this way, if the region A and the region B are molded of materials that differ in hardness, the inner diameter, the outer diameter, and the thickness of the cylindrical wall of the cylindrical component, the length of the high-kink resistance region in the circumferential direction, and the like are highly freely selected. In such a configuration, it is preferable that the cylindrical component has a plurality of the regions A and a plurality of the regions B, and the regions A and the regions B are alternately arranged along the circumferential direction of the cylindrical component because the molding stability of the cylindrical component is good.

The above hardness is, e.g., Shore hardness, and is a value measured with a durometer (type D) according to JIS Z 2246. Examples of a preferable combination of the material of the region A and the material of the region B include D70/D40 (the former is the material of the region A and the latter is the material of the region B, and the same applies to the following examples), D70/D50, D60/D40, and D50/D30.

In the medical-insert guide of an aspect of the present invention, the cylindrical component has kink resistance performance (length), which is evaluated using a loop method, of preferably 500 mm or less, and more preferably 300 mm or less. In an aspect of the present invention, the kink resistance performance is a value evaluated using the following method.

### Evaluation of Kink Resistance Performance

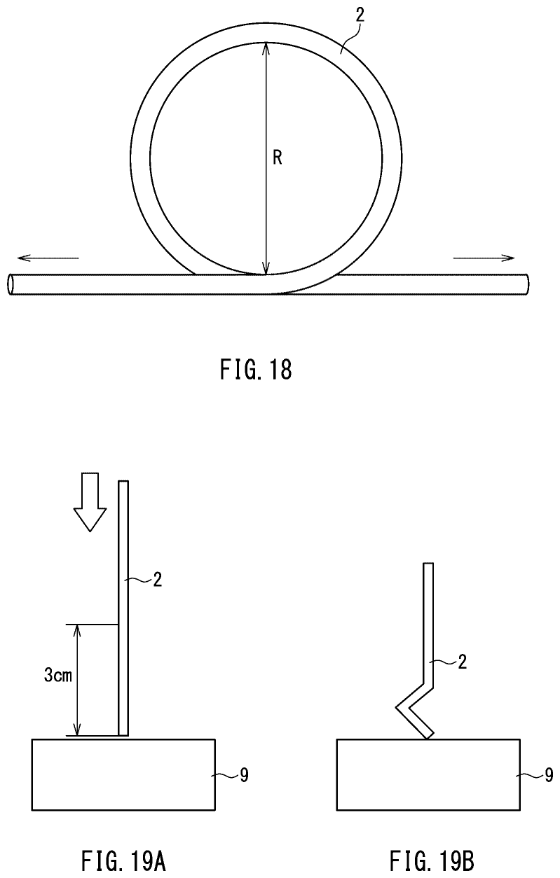

1. Two positions on the surface of the cylindrical component are marked. The distance between the pair of marks is 1,000 mm.
2. The two marks are put on each other to form a loop having a loop length of 1,000 mm and a loop inner diameter of R, as illustrated in FIG. 18.
3. Both the ends of the cylindrical component 2 are pulled in the directions of the arrows, respectively, to reduce the loop inner diameter R. At the time of occurrence of bending, the loop length (the length of the cylindrical component 2 forming the loop) is measured. The loop length is used as an indicator that represents the kink resistance performance.

The smaller the value, the more excellent the kink resistance performance.

The distance between the pair of marks may be 600 mm. If the length of the medical-insert guide of the present disclosure is shorter than 600 mm, the distance between the pair of marks is the maximum possible length.

In the medical-insert guide of an aspect of the present invention, the insertion-side front end of the cylindrical component and a portion of the cylindrical component near the insertion-side front end (which may be also referred to as “insertion-side front end portion”) have a bending strength of preferably 50 g or more, and more preferably 65 g or more.

In an aspect of the present invention, the bending strength is measured by pressing the end face of the insertion-side front end of the cylindrical component, which is held by its portion that is 3 cm from the insertion-side front end, against a balance, and the bending strength is a weight measured by the balance when the cylindrical component buckles (bends). Because the bending strength is a parameter that represents the degree of bending easiness during compression of the insertion-side front end portion in the longitudinal direction, the kink resistance of the insertion-side front end portion can be evaluated from the bending strength.

This method allows easy evaluation of the kink resistance for aspects for which the kink resistance cannot be evaluated using the loop method, such as a case where the high-kink resistance regions are present only at a portion of the cylindrical component positioned at the pharynx and the upper esophageal sphincter, and the insertion-side front end portion.

More specifically, the bending strength is a value evaluated using the following method.

### Method for Evaluating Bending Strength

1. A portion of the cylindrical component 2 that is 3 cm from the insertion-side front end is held, as illustrated in FIG. 19.
2. The insertion-side front end of the cylindrical component 2 is pressed against an electronic balance 9 while the cylindrical component 2 is held such that the axis of the cylindrical component 2 from the held portion to the front end is perpendicular to the electronic balance, and the weight is measured.
3. When the cylindrical component 2 with the insertion-side front end pressed against the electronic balance 9 buckles, the measurement is ended.
4. The maximum value of the weight is recorded.

The “maximum value of the weight” measured using the “Method for evaluating bending strength” is the bending strength. If the bending strength of the cylindrical component is approximately the same as or more than the bending strength of existing feeding catheters of 6 Fr (outer diameter: 2.0 mm) that do not cause the problem of misalignment of the front end during indwelling, misalignment of the front end of the indwelled cylindrical component is effectively suppressed. However, if the bending strength of the cylindrical component is excessively large, the patient feels more uncomfortable during insertion of the cylindrical component into the body. Thus, the bending strength is preferably 440 g or less, and more preferably 380 g or less. These upper limits of the bending strength are smaller than the bending strength (440 g) of a feeding catheter with a guide wire of 12 Fr inserted in its inner cavity because there is no effect on a living body.

In the medical-insert guide of an aspect of the present invention, the proportion of the total of the circumferential lengths of the high-kink-resistance regions to the circumferential length of the cylindrical component (100×the circumferential length of each high-kink resistance region×the number of the high-kink-resistance regions/the circumferential length of the cylindrical component) varies depending on the material of the cylindrical component, but is preferably 10 to 80% and more preferably 15 to 40% to achieve both the kink resistance and the easy deformation in the radial direction.

In the medical-insert guide of an aspect of the present invention, it is preferable that when the cylindrical component is pressed in the radial direction by a pressure of 50 mmHg, the cylindrical component has an outer diameter of 2.5 mm or less in the pressing direction. Such a configuration allows mixed ingestion, which basically orally takes nutrition (meals) and supplementarily takes a shortage of the nutrition by nasal tube feeding, to be performed with less uncomfortableness even when the medical-insert guide of an aspect of the present invention is indwelled.

From a similar reason, the cylindrical component preferably has flexibility to the degree that when it is pressed in the radial direction by a pressure of 50 mmHg, the cylindrical component is flattened and its inner cavity is eliminated. If the cylindrical component 2 pressed in the radial direction is flattened and the inner cavity is eliminated, the “outer diameter of the cylindrical component in the pressing direction” can be interpreted as the “thickness of the cylindrical component in the pressing direction”.

More specifically, as described above, the nasal tube feeding has an advantage that it can reliably and efficiently supply necessary nutrition to the stomach of the patient. However, the mouth and throat are not used, and thus the swallowing function of the patient deteriorates. Accordingly, in medical setting, before the nasal tube feeding is attempted, oral ingestion that orally takes nutrition is attempted first. However, a patient who has less wanted to eat and a patient who has dysphagia easily lapse into malnutrition.

Therefore, in medical setting, the above mixed ingestion is performed while, e.g., a feeding catheter of a small diameter of approximately 2.5 mm is indwelled to reduce uncomfortableness of the patient during oral ingestion and prevent malnutrition. Further, in recent years, some medical institutions have performed the mixed ingestion while a feeding catheter of 6.5 Fr (the diameter is approximately 2.15 mm) has been indwelled. However, such mixed ingestion is applied to only, e.g., patients whose throat sensation has declined such as stroke patients. Further, because the above feeding catheter of a small diameter is used, and indwelled for a certain period of time (e.g., two weeks), the catheter may clog.

In a preferable aspect of the medical insert guide of the present invention, when the cylindrical component is pressed in the radial direction by a pressure of 50 mmHg, the cylindrical component has an outer diameter or thickness of 2.5 mm or less in the pressing direction, as described above. Alternatively, the cylindrical component preliminarily has a flat cross-sectional shape, and has an outer diameter or thickness of 2.5 mm or less when it is not pressed. Thus, the above mixed ingestion is possible. The feeding catheter has a hardness that maintains the shape of its inner cavity when it is pressed by, e.g., the pharynx or the esophagus, and has rigidity to the degree that prevents, e.g., buckling of the feeding catheter when it is inserted into a body. By contrast, the cylindrical component that constitutes the medical-insert guide of an aspect of the present invention is easily deformed and has low elasticity, and preferably has flexibility that prevents the deformed cylindrical component from pressing the inner wall surfaces of, e.g., the pharynx and the esophagus.

Therefore, if the medical-insert guide of an aspect of the present invention is indwelled instead of a feeding catheter, the uncomfortableness during the indwelling is reduced, and thus oral ingestion with less uncomfortableness is possible even during the indwelling. Further, a feeding catheter is inserted into the medical-insert guide of an aspect of the present invention for tube nutrition administration, and after the tube administration, the feeding catheter is removed from the medical-insert guide. Thus, the feeding catheter cannot clog. Therefore, the diameter of the feeding catheter to be used is more freely selected.

On the other hand, according to “THE EFFECT OF ESOPHAGECTOMY AND/OR LARYNGECTOMY ON UPPER ESOPHAGEAL SPHINCTER FUNCTION—CLINICAL STUDY OF 3 PATIENTS WITH ESOPHAGEAL CANCER—”, Journal of Japan Surgical Association 51(4), 699-703, 1990, Taigo TOKUHARA et al. in Second Division (Chief: Professor Hiroaki KINOSHITA), Department of Surgery, Osaka City University Medical School

(https://www.jstage.jst.go.jp/article/ringe1963/51/4/51_4_699/_pdf/-char/en), the maximum closing pressure in digestive organs is the pressure by the upper esophageal sphincter, which is 0.020 N/mm² (approximately 150 mmHg). Therefore, if the cylindrical component pressed in the radial direction by a pressure of 150 mmHg has an outer diameter or thickness of preferably 2.15 mm (corresponding to 6.5 Fr) or less in the pressing direction, favorable oral ingestion is possible even while the medical-insert guide is indwelled.

The pharynx pressure or esophageal pressure during oral ingestion varies depending on the position and each individual, but has a value within a range of 50 mmHg to 300 mmHg in a position having a highest value. Therefore, if the cylindrical component pressed by a pressure of 50 mmHg (approximately 0.006 N/mm²) has an outer diameter or thickness of preferably 2.5 mm or less, and more preferably 2.15 mm or less in the pressing direction, favorable oral ingestion is possible even while the medical-insert guide is indwelled.

More specifically, the outer diameter or thickness in the pressing direction of the cylindrical component pressed by a pressure of 150 mmHg or 50 mmHg is a value evaluated using the method described in the following “Method for evaluating compressibility”.

### Method for Evaluating Compressibility

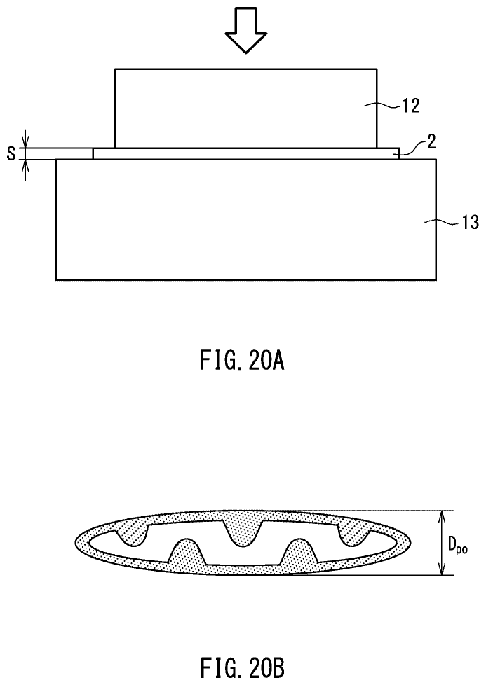

1. A load cell and an upper compression jig 12 are connected to a tensile tester. A lower compression jig 13 is positioned under the upper compression jig.
2. A cylindrical component 2 is set in a gap between the upper compression jig 12 and the lower compression jig 13, as illustrated in FIG. 20A.
3. The upper compression jig 12 is lowered at a speed of 1 mm/min, and the distance S between the upper compression jig 12 and the lower compression jig 13 is measured when the cylindrical component 2 is pressed by a pressure of 150 mmHg or 50 mmHg, that is, the outer diameter Dₚₒ or thickness in the pressing direction of the cylindrical component 2 is measured when the cylindrical component 2 is pressed by a pressure of 150 mmHg or 50 mmHg (FIG. 20B).

In the medical-insert guide of an aspect of the present invention, it is preferable that at least one of an optical fiber or a contrast line is embedded in the cylindrical wall of the cylindrical component and extends across the whole length of the cylindrical component in the longitudinal direction. Such a configuration allows the position of the front end of the indwelled medical-insert guide to be checked using a simple method.

Instead of permanent embedment of an optical fiber or a contrast line in the cylindrical wall, a medical insert guide 1 according to an aspect of the present invention may have a configuration in which the cylindrical component includes a flexible hard member that has high kink resistance and is independently present when the flexible hard member is removed from the cylindrical wall of the cylindrical component, and the flexible hard member can be removably inserted in the cylindrical wall.

That is, in the medical-insert guide of an aspect of the present invention, the cylindrical component has at least two high-kink resistance regions, each high-kink-resistance region extends in the longitudinal direction of the cylindrical component, and is less likely to be subjected to compression deformation in the longitudinal direction than a region of the cylindrical component that is adjacent to the high-kink-resistance region in the circumferential direction is likely to be subjected thereto, the cylindrical component includes the flexible hard member, the flexible hard member is removably inserted in the cylindrical wall along the longitudinal direction, is independently present when it is removed from the cylindrical wall, and has kink resistance, at least one of the at least two high-kink-resistance regions is formed integrally with the adjacent region when the cylindrical component is molded, and at least one of the at least two high-kink resistance regions includes the flexible hard member that has been removably inserted in the cylindrical wall along the longitudinal direction. In the present aspect, while the flexible hard member is inserted in the cylindrical wall of the cylindrical component, the portion of the cylindrical component where the flexible hard member is inserted functions as the high-kink resistance region.

In the present aspect, the number of the portions where the flexible hard member has been inserted is at least one fewer than the total number of the high-kink-resistance regions of the cylindrical component. Thus, even after all the flexible hard members are removed, the cylindrical component has at least one high-kink-resistance region made of resin material that constitutes the cylindrical component, such as the thick portion, and thus misalignment of the front end of the indwelled cylindrical component is suppressed without increasing the uncomfortableness during the indwelling.

As described above, the aspect in which the portion of the cylindrical component where the flexible hard member has been inserted is the high-kink-resistance region, and the flexible hard member can be removably inserted in the cylindrical wall of the cylindrical component is preferable because the uncomfortableness during indwelling of the medical-insert guide is further reduced.

More specifically, when the medical-insert guide including the cylindrical component of such an aspect is inserted into the body of a patient, the medical-insert guide includes the flexible hard member inserted in the cylindrical wall. After the insertion, the flexible hard member is removed from the cylindrical wall, and thus the number of the high-kink resistance regions in the cylindrical component can be reduced. Further, even after all the flexible hard members are removed from the cylindrical wall, the cylindrical component has at least one high-kink resistance region made of resin material, as described above, and thus misalignment of the front end of the indwelled cylindrical component is suppressed.

The flexible hard member may be an optical fiber or a member containing a contrast medium. Before a meal of the patient, the flexible hard member may be inserted again into the cylindrical wall to check occurrence of misalignment of the insertion-side front end of the cylindrical component by means of the flexible hard member.

The cylindrical component of the medical-insert guide of an aspect of the present invention has an outer diameter of preferably 15.0 mm or less, and more preferably 7.3 mm or less because the insertion into a nostril is easy and the uncomfortableness caused by the indwelling is reduced. The lower limit of the outer diameter of the cylindrical component 2 is not limited, but is preferably 1.2 mm or more, more preferably 1.9 mm or more, and further preferably 2.9 mm or more to allow the inner diameter that enables easy insertion and removal of a feeding catheter to be set.

The cylindrical wall of the cylindrical component of the medical-insert guide of an aspect of the present invention (that is the thin portion in an embodiment where the thick portion is the high-kink resistance region) has a thickness of preferably 0.6 mm or less, and more preferably 0.4 mm or less because the cylindrical component is easily deformed by pharynx pressure or esophageal pressure, and the uncomfortableness during the indwelling is reduced, and also preferably 0.05 mm or more, and more preferably 0.075 mm or more considering the strength. Considering the moldability, the thickness is also preferably 0.05 mm or more, and more preferably 0.075 mm or more.

A feeding catheter of preferably 3 to 16 Fr (outer diameter: 1.0 to 5.3 mm), and more preferably 5 to 12 Fr (outer diameter: 1.7 to 4.0 mm) can be removably inserted into the inner cavity of the cylindrical component of the medical insert guide of an aspect of the present invention. The inner diameter of the cylindrical component (in an embodiment where the cylindrical component is provided with a plurality of the high-kink resistance regions by forming a plurality of protruding rows that protrude toward the inner cavity, the inner diameter is the diameter of an imaginary circle that connects the tops of the protruding rows) is preferably 1.1 mm or more, and more preferably 1.8 mm or more considering a sufficiently high supply rate of nutrition by tube administration and easy insertion and removal of a feeding catheter, and also is preferably 14.9 mm or less, and more preferably 7.2 mm or less to allow the outer diameter that reduces the uncomfortableness caused by the indwelling to be set.

The material of the cylindrical component of the medical-insert guide of an aspect of the present invention is not limited, but is, e.g., at least one selected from the group consisting of polyethylene, polyvinyl chloride, polypropylene, polyethylene terephthalate, polytetrafluoroethylene, hexafluoropropylene, vinylidene fluoride, polybutadiene, polyurethane, polystyrene-based thermoplastic elastomers, urethane-based thermoplastic elastomers, and polyolefin-based thermoplastic elastomers. The cylindrical component is indwelled in the body of a patient, and may be in contact with body liquids of the patient, especially, gastric juice and intestinal juice. Thus, polyethylene, polypropylene, polybutadiene, and polyurethane, which are less likely to be changed in properties (e.g., curing) by being in contact with the liquids, are preferable, for example.

Hereinafter, the present invention will be described in detail by way of preferable embodiments. However, the present invention is not limited to the following embodiments.

### Embodiment 1

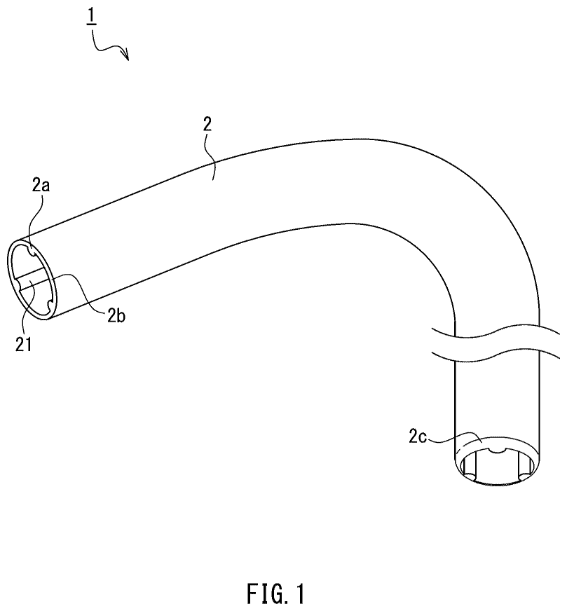

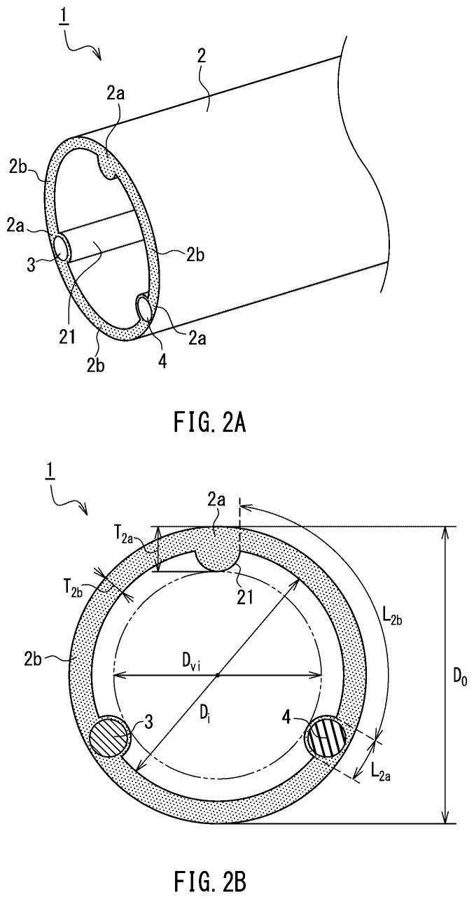

FIG. 1 is a perspective view of a medical-insert guide 1 according to an aspect of the present invention. FIGS. 2A and 2B are an enlarged view and a cross-sectional view of its base-side end portion.

The medical-insert guide 1 includes a cylindrical component 2 whose cross section has a circular outer shape. As is clear from FIG. 1, the insertion-side front end of the cylindrical component 2 has a curved face 2c not to injure, e.g., the gastrointestinal tract. The cylindrical component 2 is easily deformed by being pressed in the radial direction and has low elasticity, and thus has flexibility that allows its cross-sectional shape to be easily deformed into a flat shape by, e.g., pharynx pressure or esophageal pressure, and prevents the deformed cross-sectional shape from pressing the internal wall surfaces of, e.g., the pharynx and the esophagus. Thus, the uncomfortableness during the indwelling is less.

As can been seen from the cross section of the cylindrical component 2, the inner face is uneven. The cylindrical component 2 has three protruding rows 21 each protruding toward the inner cavity of the cylindrical component 2 and extending across the whole length of the cylindrical component 2 in the longitudinal (length) direction. The protruding rows 21 are formed at predetermined intervals along the circumferential direction of the cylindrical component 2.

In the cylindrical component 2, the three protruding rows 21 are arranged at predetermined intervals, e.g., at regular intervals, along the circumferential direction. Thus, in the cylindrical component 2, thick portions 2a where the cylindrical wall is relatively thick and thin portions 2b where the cylindrical wall is relatively thin are alternately arranged along the circumferential direction. Because the cylindrical wall of the thick portions 2a is thicker than that of the thin portions 2b, the thick portions 2a are less likely to be subjected to compression deformation in the longitudinal direction than the thin portions 2b are likely to be subjected thereto. Each thick portion 2a is a high-kink resistance region 2a that is less likely to be subjected to compression deformation in the longitudinal direction than a region adjacent to the thick portion 2a in the circumferential direction (the thin portion 2b) is likely to be subjected thereto. Each adjacent region (thin portion 2b) can be also referred to as a low-kink-resistance region 2b that is less likely to be subjected to compression deformation in the longitudinal direction than the thick portions 2a are likely to be subjected thereto.

As illustrated in FIG. 2B, in the medical-insert guide 1 of the present aspect, for example, the outer diameter Dₒ of the cylindrical component 2 is 4.8 mm, the diameter Dᵢ of an imaginary circle that connects the inner face of the cylindrical component 2 at the thin portions 2b is 4.2 mm, and the diameter Dᵥᵢ of an imaginary circle that connects the tops of the protruding rows 21 is 3.2 mm. The medical-insert guide 1 of the present aspect allows smooth insertion and removal of a feeding catheter of 8 Fr (outer diameter: 2.7 mm).

The cylindrical wall of the cylindrical component 2 has a thickness T₂ₐ of 0.8 mm in each thick portion 2a (high-kink-resistance region 2a), and a thickness T_{2b} of 0.3 mm in each thin portion 2b. L₂ₐ is 0.8 mm and L_{2b} is 4.2 mm. Thus, the proportion of the total of the circumferential lengths of the high-kink resistance regions 2a to the circumferential length (outer circumferential length) of the cylindrical component 2 is 16%.

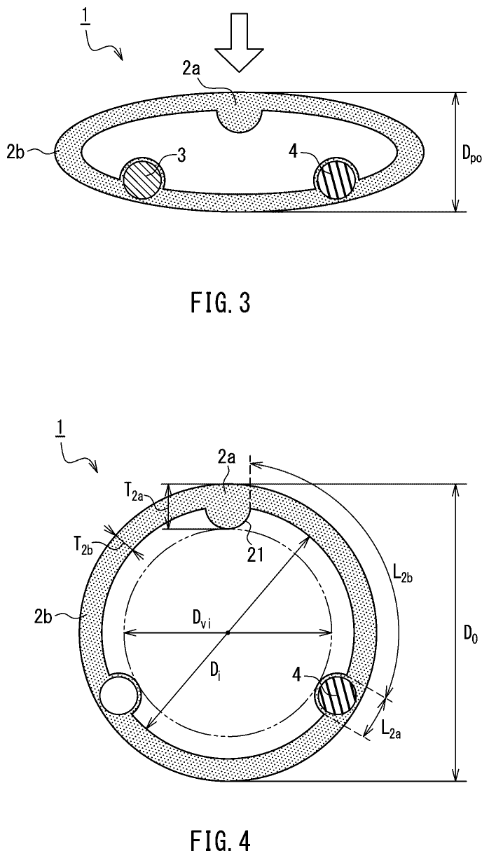

As illustrated in FIGS. 3 and 20, when the cylindrical component 2 is pressed in the radial direction by a load of 50 mmHg according to the above “Method for evaluating compressibility”, the outer diameter D_{po o}f the cylindrical component 2 in the pressing direction is 2.5 mm or less, more specifically 1.7 mm, and when the cylindrical component 2 is pressed in the radial direction by a pressure of 150 mmHg according to the above “Method for evaluating compressibility”, the outer diameter Dₚₒ of the cylindrical component 2 in the pressing direction is 1.27 mm. The bending strength of the insertion-side front end portion of the cylindrical component 2 measured according to the above “Method for evaluating bending strength” is 58.2 g. Thus, the medical-insert guide 1 of the present aspect can effectively suppress misalignment of the front end of the indwelled cylindrical component without increasing the uncomfortableness during the indwelling.

The kink resistance performance (loop length) described with reference to FIG. 18 is preferably 500 mm or less, more specifically 30 mm. One of the factors of movement of the front end of the indwelled medical-insert guide from a desired position is the bending of the indwelled medical-insert guide in the axial direction. In the body from the nose or the mouth to the inside of the gastrointestinal tract, the bending of the cylindrical component is likely to occur from the nostril to the pharynx where there is a sharp curve, especially, at the soft palate portion. When the kink resistance performance (loop length) is 500 mm or less, the bending of the cylindrical component is effectively suppressed.

In the medical-insert guide 1 according to an aspect of the present invention, the cylindrical component 2 has the high-kink resistance regions 2a that extend across the whole length of the cylindrical component 2 in the longitudinal direction and are less likely to be subjected to compression deformation in the longitudinal direction than the adjacent regions are likely to be subjected thereto. This configuration suppresses bending of the cylindrical component 2 caused by, e.g., peristalsis of organs such as the esophagus, and the friction against a medical insert, such as a feeding catheter, inserted into or removed from the cylindrical component 2. Thus, the front end of the cylindrical component 2 that has been positioned with, e.g., a camera at the time of indwelling is less likely to excessively retreat from the position.

Further, when an expected pressing force such as pharynx pressure or esophageal pressure is applied to the medical-insert guide 1 in the radial direction, the thin portions 2b are more likely to be bent than the thick portions 2a (high-kink-resistance regions 2a) are likely to be bent, and thus the outer shape of the cross section of the cylindrical component 2 deforms into a flat shape, as illustrated in FIG. 3. The cylindrical component does not press the inner wall surfaces of, e.g., the pharynx and the esophagus during indwelling. Therefore, the uncomfortableness during the indwelling can be effectively suppressed, and oral nutrition ingestion is possible while the medical-insert guide 1 is indwelled.

An optical fiber 3 and a contrast line 4 are embedded in the thick portions 2a (high-kink-resistance regions 2a) of the cylindrical component 2. As the contrast line, for example, a thermoplastic resin (e.g., polypropylene) containing a radiopaque agent of barium sulfate is shaped like a line.

Because the cylindrical component 2 of the present aspect has the protruding rows 21, the optical fiber 3 and the contrast line 4 are easily embedded in the cylindrical wall. The optical fiber 3 and the contrast line 4 each extend from the base-side end to the front end of the cylindrical component 2. The optical fiber 3 and the contrast line 4 can be embedded in the cylindrical component 2 using, e.g., conventional methods. If the base-side end of the optical fiber 3 is irradiated with light from a separately provided light source, the light propagates to the front end. Thus, it is easy to check whether the front end of the medical-insert guide 1 is positioned at a desired position. Further, because the contrast line 4 is embedded, radiography can be used to check whether the front end of the medical-insert guide 1 is positioned at a desired position.

The optical fiber 3 may be capable of being removably inserted into the cylindrical component 2. FIG. 4 shows the cylindrical component 2 from which the optical fiber 3 has been removed. In this way, if the optical fiber 3 can be removed from the cylindrical component 2, the number of the high-kink resistance regions 2a can be reduced by removing the optical fiber 3 from the cylindrical component 2 after the medical-insert guide 1 has been inserted in a predetermined position in a body. This configuration is preferable because the uncomfortableness during indwelling of the medical-insert guide 1 can be reduced.

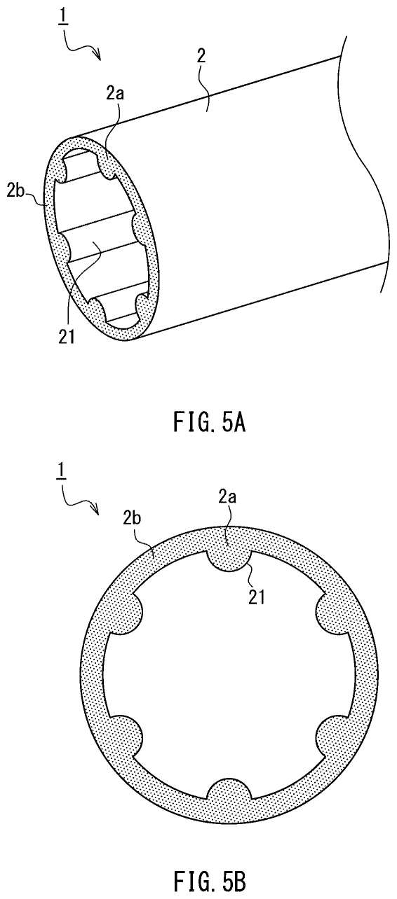

Although in the medical-insert guide 1 described with reference to FIGS. 1 to 3, the cylindrical component 2 has the three protruding rows 21 protruding toward the inner cavity, and has the three thick portions 2a (high-kink-resistance regions) and the three thin portions 2b (low-kink-resistance regions), the number of the thick portions 2a and the number of the thin portions 2b are not limited. For example, the cylindrical component 2 may have six thick portions 2a and six thin portions 2b, as illustrated in FIGS. 5A and 5B. The number of the thick portions 2a, the number of the thin portions 2b, the proportion of the circumferential length of the thick portions 2a to the circumferential length of the cylindrical component 2, and the proportion of the circumferential length of the thin portions 2b to the circumferential length of the cylindrical component 2 are appropriately selected considering both the kink resistance and the easiness of deformation into a flat shape. Arrangement of the thick portions 2a and the thin portions 2b at regular intervals along the circumferential direction of the cylindrical component 2 is preferable considering molding stability but is not essential. The protruding rows 21 may protrude toward the outside of the cylindrical component 2. Alternatively, the cylindrical component 2 may have both protruding rows protruding toward the inner cavity, and protruding rows protruding toward the outside, and they may form the thick portions 2a. The optical fiber 3 and the contrast line 4 may be eliminated.

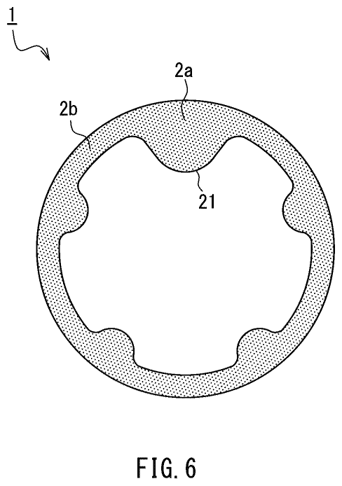

The surface of the protruding row 21 is preferably but is not limited to a curved surface that is highest in its central portion. In the cross section of the cylindrical component 2 cut in a direction perpendicular to the longitudinal direction, the cross-sectional shape of the protruding row may be any one of, e.g., a semicircular shape, a semielliptical shape, a triangular shape, or a trapezoidal shape. If the cylindrical component has a plurality of protruding rows, the cross-sectional shapes of all the protruding rows do not necessarily have the same shape or the same size. For example, the cross-sectional shapes may be at least two selected from the group consisting of a semicircular shape, a semielliptical shape, a triangular shape, and a trapezoidal shape. For example, one of five protruding rows 21 may have a larger cross-sectional shape than that of the other four protruding rows 21, as illustrated in FIG. 6.

### Embodiment 2

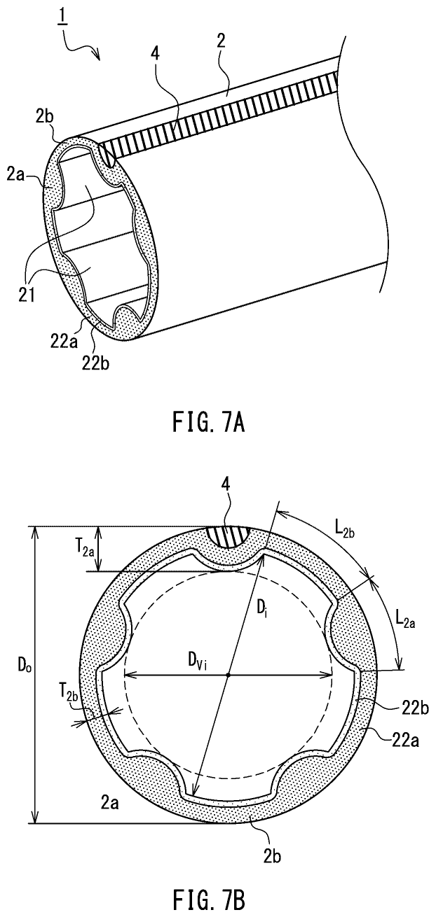

FIGS. 7A and 7B are an enlarged view and a cross-sectional view of a base-side end portion of a medical-insert guide 1 according to an aspect of the present invention.

The medical-insert guide 1 of the present aspect is the same as the medical-insert guide 1 of Embodiment 1 except that a cylindrical component 2 is a tube of a multi-layered structure, the cylindrical component includes a first layer 22a, and a second layer 22b that is innermost and has an inner face that constitutes the inner circumferential face of the cylindrical component 2, a contrast line 4 is exposed to the outside of the cylindrical component 2, and the number of thick portions 2a (high-kink-resistance regions) and the number of thin portions 2b (low-kink-resistance regions) are each five.

The first layer 22a is made of a material of which a tube that has a higher flexibility and a higher kink resistance than those of the second layer 22b can be molded. The material is preferably a diene-based polymer. Examples of the diene-based polymer include 1,4-polybutadiene, 1,2-polybutadiene, polyisoprene, thermoplastic polyurethane, polyvinyl chloride, styrene-butadiene block copolymers, various elastomers. They can be used alone or in combination of two or more. Among them, 1,4-polybutadiene or 1,2-polybutadiene is preferable, and 1,2-polybutadiene is much preferable considering the flexibility, kink resistance, and moldability.

The second layer 22b is made of a material whose sliding resistance between the material and, e.g., a catheter inserted into and removed from the inner cavity of the cylindrical component 2 is smaller than the sliding resistance between the material of the first layer 22a and the catheter. The material is preferably an olefin-based polymer. Examples of the olefin-based polymer include polyethylene such as high-density polyethylene (HDPE), linear low density polyethylene (LLDPE), low-density polyethylene (LDPE), very-low-density polyethylene (VLDPE), and ultra-low-density polyethylene (ULDPE), ethylene-propylene copolymers, ethylene vinyl alcohol copolymers, and ethylene-methyl (meth)acrylate copolymers. They can be used alone or in combination of two or more. Among them, for example, the linear low density polyethylene (LLDPE) or the high-density polyethylene (HDPE) or a mixture of them has a smaller sliding resistance, and a higher joining strength between it and an adjacent layer due to its thermal fusion bonding.

Because in the present aspect, the cylindrical component 2 includes the first layer 22a made of a material that has a higher flexibility and a higher kink resistance, and the second layer 22b as the innermost layer made of a material having a smaller sliding resistance between the material and the medical insert, the uncomfortableness during the indwelling can be suppressed, misalignment of the front end of the cylindrical component can be suppressed, and medical inserts can be easily inserted and removed. The cylindrical component 2 having such a layered structure can be manufactured using conventional molding techniques. For example, the cylindrical component 2 can be manufactured by simultaneously extruding the materials of the layers, and then cooling the extruded materials.

In FIG. 7, although the contrast line 4 is exposed to the outside of the cylindrical component 2 and constitutes part of the outer circumferential face of the medical-insert guide 1, the contrast line 4 is not limited to this configuration. The whole contrast line 4 may be embedded in the cylindrical wall of the cylindrical component 2, as in the aspect in FIG. 2. The contrast line 4 can be eliminated. In this case, a contrast agent may be blended with the innermost layer 22b.

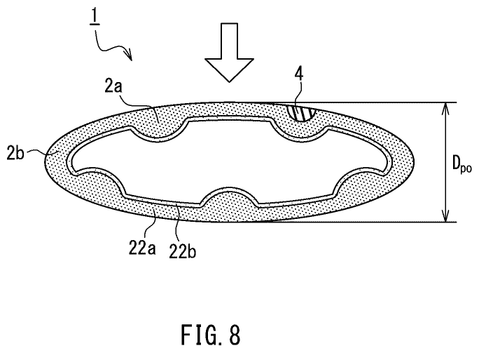

In FIGS. 7 and 8, the cylindrical component 2 has the double-layered structure including the first layer 22a and the second layer 22b, and the first layer 22a is thicker than the second layer 22b (innermost layer). However, the thickness of each layer and the number of the layers are not limited to this example as long as the cylindrical component 2 has flexibility, a high kink resistance, and a small sliding resistance between the cylindrical component 2 and the medical insert.

In the medical-insert guide 1 of the present aspect, for example, the cylindrical component 2 has the five protruding rows 21 protruding toward the inner cavity, and has the five thick portions 2a (high-kink-resistance regions) and the five thin portions 2b (low-kink-resistance regions), as illustrated in FIGS. 7A and 7B. The first layer 22 a is made of 1,2-polybutadiene, and the second layer 22b is made of linear low-density polyethylene (LLDPE). As illustrated in FIG. 7B, the outer diameter Dₒ of the cylindrical component 2 is 5.23 mm, the diameter Dᵢ of an imaginary circle that connects the inner face of the cylindrical component 2 at the thin portions 2b is 4.87 mm, and the diameter Dᵥᵢ of an imaginary circle that connects the tops of the protruding rows 21 is 4.23 mm, for example. The medical-insert guide 1 of the present aspect allows smooth insertion and removal of a feeding catheter of 8 Fr (outer diameter: 2.7 mm).

The cylindrical wall of the cylindrical component 2 has a thickness T₂ₐ of 0.5 mm in each thick portion 2a (high-kink-resistance region 2a), and a thickness T_{2b} of 0.18 mm in each thin portion 2 b. L₂ₐ is 0.65 mm and L_{2b} is 2.00 mm. Thus, the proportion of the total of the circumferential lengths of the high-kink-resistance regions 2a to the circumferential length (outer circumferential length) of the cylindrical component 2 is 25%.

As illustrated in FIG. 8, when the cylindrical component 2 is pressed in the radial direction by a pressure of 50 mmHg according to the above “Method for evaluating compressibility”, the outer diameter Dₚₒ of the cylindrical component 2 in the pressing direction is 2.5 mm or less, more specifically 2.3 mm, and when the cylindrical component 2 is pressed in the radial direction by a pressure of 150 mmHg according to the above “Method for evaluating compressibility”, the outer diameter Dₚₒ of the cylindrical component 2 in the pressing direction is 1.14 mm. The bending strength of the insertion-side front end portion of the cylindrical component 2 measured according to the above “Method for evaluating bending strength” is 75 g. The medical-insert guide 1 of the present aspect can effectively suppress misalignment of the front end of the indwelled cylindrical component without increasing the uncomfortableness during the indwelling.

The kink resistance performance (loop length) described with reference to FIG. 18 is 500 mm or less, more specifically 340 mm. Further, even if the cylindrical component 2 is held by both the longitudinal ends, and, for example, pulled a plurality of times, or twisted a plurality of times, interlayer stripping does not occur.

In the medical-insert guide 1 of the present aspect, the cylindrical component 2 has the high-kink resistance regions 2a that extend across the whole length of the cylindrical component 2 in the longitudinal direction and are less likely to be subjected to compression deformation in the longitudinal direction than the adjacent regions are likely to be subjected thereto, similarly to Embodiment 1. Thus, the front end of the cylindrical component 2 that has been positioned in a body is less likely to excessively retreat from the position.

Further, when an expected pressing force such as pharynx pressure or esophageal pressure is applied to the medical-insert guide 1 in the radial direction, the thin portions 2b are more likely to be bent than the thick portions 2a (high-kink-resistance regions 2a) are likely to be bent, and thus the outer shape of the cross section of the cylindrical component 2 deforms into a flat shape, as illustrated in FIG. 8. The cylindrical component does not press the inner wall surfaces of, e.g., the pharynx and the esophagus during indwelling. Therefore, the uncomfortableness during the indwelling can be effectively suppressed, and oral nutrition ingestion is possible while the medical-insert guide 1 is indwelled.

### Embodiment 3

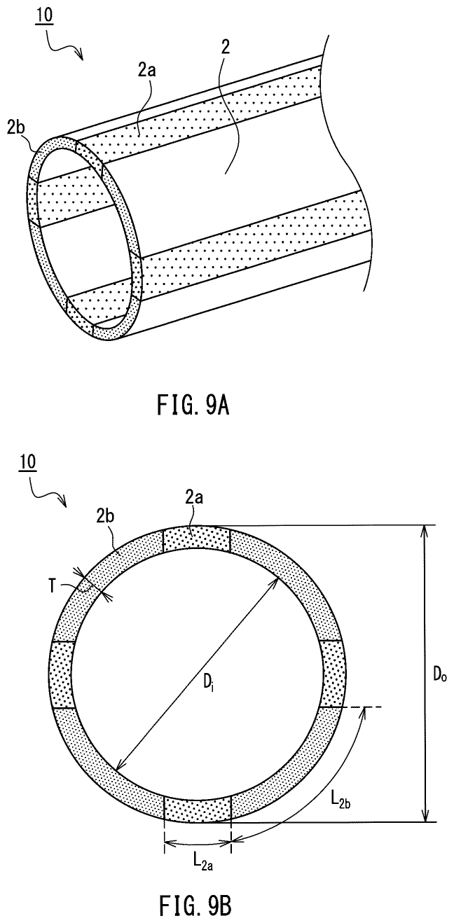

FIGS. 9A and 9B are an enlarged view and a cross-sectional view of the base-side end portion of a medical-insert guide 10 according to an aspect of the present invention. The medical-insert guide 10 of the present aspect is different from both Embodiments 1 and 2 in that a cylindrical component 2 is made of two types of materials that differ in hardness and are alternately arranged in the circumferential direction.

The medical-insert guide 10 of the present aspect includes the cylindrical component 2 whose cross section has a circular outer shape. The cylindrical component 2 is easily deformed by being pressed in the radial direction and has low elasticity, similarly to Embodiment 1, and thus has flexibility that allows its cross-sectional shape to be easily deformed into a flat shape by, e.g., pharynx pressure or esophageal pressure, and prevents the deformed cross-sectional shape from pressing the internal wall surfaces of, e.g., the pharynx and the esophagus. Thus, the uncomfortableness during indwelling is less.

The medical-insert guide 10 of the present aspect is different from Embodiment 1 in that the cylindrical component 2 has a plurality of regions A2a made of a material having a relatively high hardness and a plurality of regions B2b made of a material having a relatively low hardness, the regions A2a and the regions B2b are alternately arranged in the circumferential direction, and the thickness of the cylindrical wall of the cylindrical component 2 is constant in the circumferential direction. The regions A2a are less likely to be subjected to compression deformation in the longitudinal direction than the regions B2b are likely to be subjected thereto because the regions A2a are made of a material having a higher hardness than the hardness of the material of the regions B2b. The regions A2a are high-kink-resistance regions 2a that are less likely to be subjected to compression deformation in the longitudinal direction than regions adjacent to the regions A2a in the circumferential direction (the regions B2b) are likely to be subjected thereto. The adjacent regions (regions B2b) can be also referred to as low-kink-resistance regions 2b that are more likely to be subjected to compression deformation in the longitudinal direction than the regions A2a are likely to be subjected thereto.

In the medical-insert guide 10 of the present aspect, for example, the cylindrical component 2 has an outer diameter Dₒ of 4.0 mm and an inner diameter Dᵢ of 3.2 mm, as illustrated in FIG. 9B. The medical-insert guide 10 of the present aspect allows smooth insertion and removal of a feeding catheter of 8 Fr (outer diameter: 2.7 mm).

The cylindrical wall of the cylindrical component 2 has a thickness T of 0.4 mm. L₂ₐ is 0.6 mm and L_{2b} is 2.5 mm, for example. The proportion of the total of the circumferential lengths of the high-kink resistance regions 2a to the circumferential length (the length of the outer circumference) of the cylindrical component 2 is 19%.

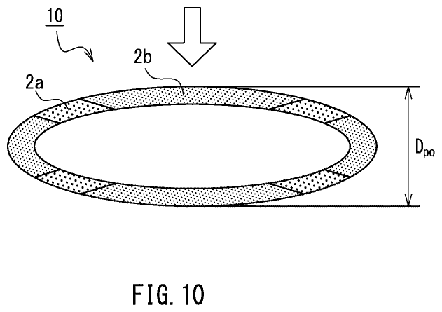

As illustrated in FIG. 10, when the cylindrical component 2 is pressed in the radial direction by a pressure of 50 mmHg according to the above “Method for evaluating compressibility”, the outer diameter Dₚₒ of the cylindrical component 2 in the pressing direction is 2.5 mm or less, and when the cylindrical component 2 is pressed in the radial direction by a pressure of 150 mmHg according to the above “Method for evaluating compressibility”, the outer diameter Dₚₒ of the cylindrical component 2 in the pressing direction is 2.15 mm or less. The bending strength of the insertion-side front end portion described with reference to FIG. 19 is 440 g or less.

The kink resistance performance (loop length) described with reference to FIG. 18 is 500 mm or less.

In the medical-insert guide 10 of the present aspect, the cylindrical component 2 also has the high-kink resistance regions 2a that extend across the whole length of the cylindrical component 2 in the longitudinal direction and are less likely to be subjected to compression deformation in the longitudinal direction than the adjacent regions are likely to be subjected thereto, similarly to Embodiment 1. Thus, the front end of the cylindrical component 2 that has been positioned in a body is less likely to excessively retreat from the position.

Further, when an expected pressing force such as pharynx pressure or esophageal pressure is applied to the medical-insert guide 10 in the radial direction, the regions B2b are more likely to be bent than the regions A2a (high-kink-resistance regions 2a) are likely to be bent, and thus the outer shape of the cross section of the cylindrical component 2 deforms into a flat shape, as illustrated in FIG. 10. Therefore, the uncomfortableness during the indwelling can be effectively suppressed, and oral nutrition ingestion is possible while the medical-insert guide 10 is indwelled.

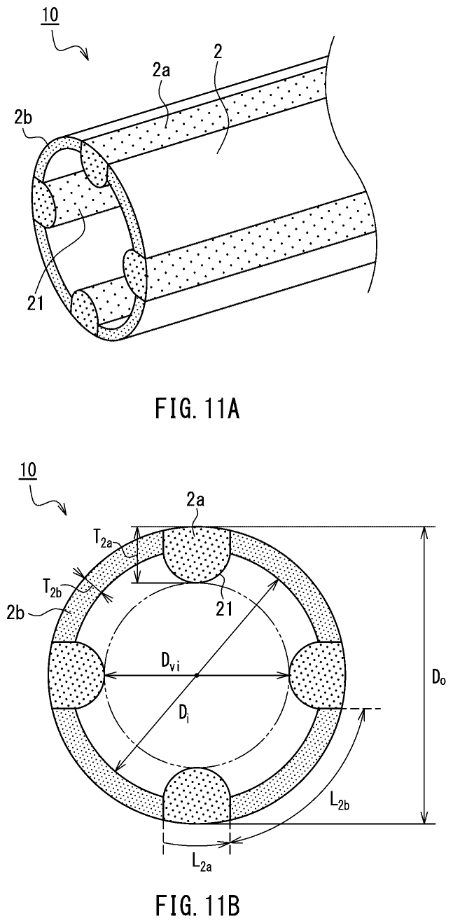

In the medical-insert guide 10 described with reference to FIGS. 9 and 10, the cylindrical wall of the cylindrical component 2 has a thickness that is constant in the circumferential direction. However, the thickness of the cylindrical wall in each region A2a (high-kink resistance region 2a) may be larger than the thickness of the cylindrical wall in each region B2b, as illustrated in FIG. 11. Such a configuration is advantageous in the control of the kink resistance, easy deformation in the radial direction, and deformation into a flat shape.

In the medical-insert guide 10 illustrated in FIG. 11, the cylindrical component 2 has an outer diameter Dₒ of 4.8 mm, Dᵢ of, e.g., 4.0 mm, and Dᵥᵢ of, e.g., 3.2 mm. The medical-insert guide 10 allows smooth insertion and removal of a feeding catheter of 8 Fr (outer diameter: 2.7 mm).

T₂ₐ is 0.8 mm and T_{2b} is 0.4 mm. L₂ₐ is 0.8 mm and L_{2b} is 2.5 mm. The proportion of the total of the circumferential lengths of the high-kink-resistance regions 2a to the circumferential length (the length of the outer circumference) of the cylindrical component 2 is 19%. The kink resistance performance (loop length) described with reference to FIG. 18 is 500 mm or less.

### Embodiment 4

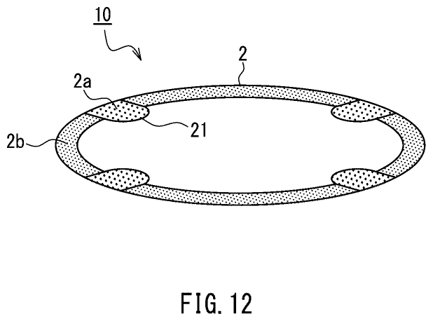

FIG. 12 is a cross-sectional view of a medical-insert guide 10 according to an aspect of the present invention. The medical-insert guide 10 of the present aspect is the same as the medical-insert guide 10 illustrated in FIG. 11 except that the cylindrical component not being pressed has an elliptical cross-sectional shape. The medical-insert guide that preliminarily has a flat cross-sectional shape can be made by, e.g., selecting the thickness, material, and the like of thin portions 2b so that the thin portions 2b can be bent by the weight of thick portions 2a even when the cylindrical component is not pressed. In the medical-insert guide of the present aspect including the cylindrical component having a flat cross-sectional shape, the cylindrical component may be made of a single material, as in the example illustrated in FIG. 5, or may have a layered structure, similarly to Embodiment 2.

### Embodiment 5

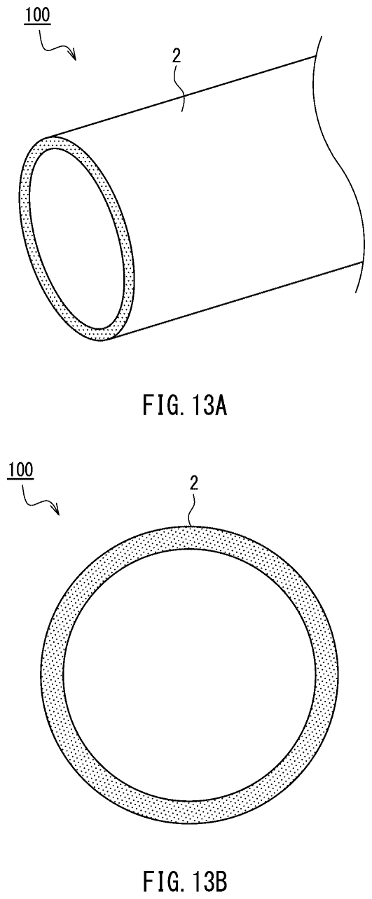

FIGS. 13A and 13B are an enlarged view and a cross-sectional view of the base-side end portion of a medical-insert guide 100 according to an aspect of the present invention.

The medical-insert guide 100 of the present aspect includes a cylindrical component 2 whose cross section has a circular outer shape. The cylindrical wall of the cylindrical component 2 has a thickness that is constant in the circumferential direction. The cylindrical component 2 is different from Embodiment 1 because the whole cylindrical component 2 is made of a single material, and thus has a kink resistance feature that is uniform in the circumferential direction.

For example, the cylindrical component 2 has an outer diameter of 4.0 mm, and the cylindrical wall has a thickness of 0.4 mm. The medical-insert guide 100 of the present aspect allows smooth insertion and removal of a feeding catheter of 8 Fr (outer diameter: 2.7 mm).

For example, the cylindrical component 2 has kink resistance performance (length), which is evaluated using the loop method, of 500 mm or less, and is molded of a material that allows easy deformation of the circular cross section of the inner cavity of the cylindrical component 2 into a flat shape when the cylindrical component 2 is indwelled in a body and is pressed by, e.g., pharynx pressure or esophageal pressure. Thus, when the medical insert guide 100 is indwelled in a body, the front end of the cylindrical component 2 that has been positioned is less likely to excessively retreat from the position, and the uncomfortableness during the indwelling is reduced.

If the cylindrical component 2 has the kink resistance feature that is uniform in the circumferential direction, as in the present aspect, a medical insert inserted into and removed from the inner cavity of the cylindrical component 2 has an outer diameter that varies depending on the inner diameter of the cylindrical component 2 but is preferably 1.7 to 4.0 mm, more specifically, feeding catheters of 5 to 12 Fr (outer diameter: 1.7 to 4.0 mm) are preferable.

The cylindrical component 2 of the medical-insert guide 100 of the present aspect may be made of a single material, as illustrated in FIG. 13, or may have a layered structure, similarly to Embodiment 2.

In any one of Embodiments 1 to 5, the cylindrical component has at least one high-kink resistance region that extends across the whole length of the cylindrical component in the longitudinal direction. In the present invention, however, the cylindrical component may have a high-kink resistance region in part of the whole length of the cylindrical component in the longitudinal direction.

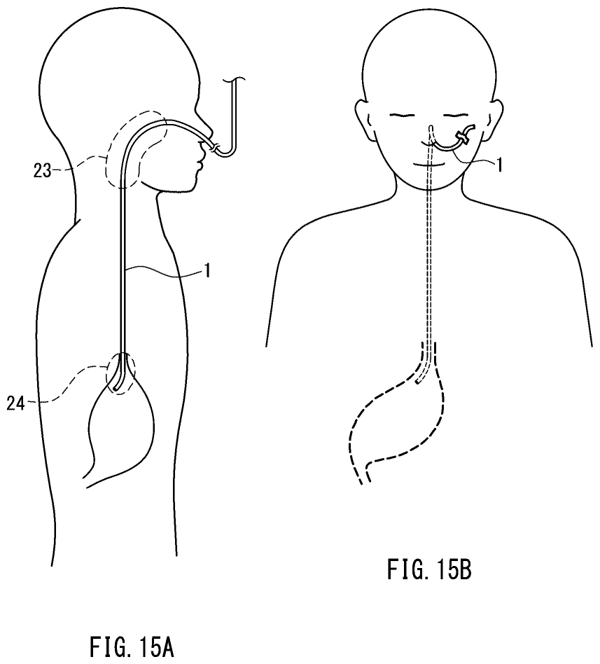

More specifically, in one aspect, a portion of the cylindrical component indwelled in a body has a high-kink resistance region and a portion thereof outside the body (see FIG. 15B) does not have a high-kink resistance region.

In another aspect, only a portion 23 of the cylindrical component positioned at the pharynx and the upper esophageal sphincter when the cylindrical component is indwelled in a body, and an insertion-side front end portion 24 of the cylindrical component may each have a high-kink resistance region that extends in the longitudinal direction of the cylindrical component (see FIG. 15A). Because the high-kink-resistance region is positioned at the pharynx and the upper esophageal sphincter that greatly deform when the patient swallows, it is possible to effectively suppress misalignment of the front end of the indwelled cylindrical component. Similarly, because the insertion-side front end portion 24 of the cylindrical component has the high-kink resistance region, misalignment of the front end of the indwelled cylindrical component is less likely to be caused by, e.g., peristalsis of the stomach or the intestines, or gastroesophageal reflux disease (GERD).

The insertion-side front end portion 24 means, for example, a portion of the cylindrical component from the insertion-side front end to approximately 3 to 15 cm from the insertion-side front end. The cylindrical component having the high-kink-resistance region in part of the whole length of the cylindrical component in the longitudinal direction can be manufactured using conventional molding techniques.

For example, the cylindrical component can be manufactured by appropriately changing the molding conditions and material blending in one molding process.

In any one of the medical-insert guides illustrated in FIGS. 1 to 3, 4, 5, 6, and 9 to 13, the cylindrical component 2 has the single-layered structure, but the inner circumferential face of the cylindrical component may be coated with silicone to reduce the sliding resistance between the medical-insert guide and the medical insert.

### Method for Using Medical-Insert Guide

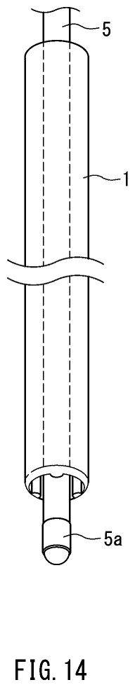

A method for using the medical-insert guide of an aspect of the present invention will be described with reference to FIGS. 14 to 17. In the following example of a method for using the medical-insert guide, the medical-insert guide 1 of Embodiment 1 is used for the explanation.

As illustrated in FIG. 14, first, a camera-including shaft 5 is inserted into the inner cavity of the medical insert guide 1. The camera-including shaft 5 is for bringing the front end of the medical insert guide 1 to a target position. For the purpose, the shaft portion of the camera including shaft 5 is flexible but is more rigid than the cylindrical component 2 of the medical-insert guide 1, and thus has rigidity to the degree that prevents, e.g., the shaft portion from buckling when it is inserted into a body. For example, the camera including shaft 5 includes a camera 5a and a light-emitting diode (LED) light (not illustrated) at its insertion start side front end.

Next, the medical-insert guide 1 with the camera including shaft 5 inserted in its inner cavity is inserted into the body of a patient from the nose. After it is confirmed with the camera that the front end has reached, e.g., the inside of the stomach, the camera-including shaft 5 is removed from the medical-insert guide 1, and only the medical-insert guide 1 is indwelled in the body, as illustrated in FIG. 15A. The portion of the medical-insert guide 1 that sticks out of the body from the nose is stuck to the face of the patient with, e.g., a piece of tape, as illustrated in FIG. 15B. In this state, the patient orally takes breakfast, lunch, and supper.

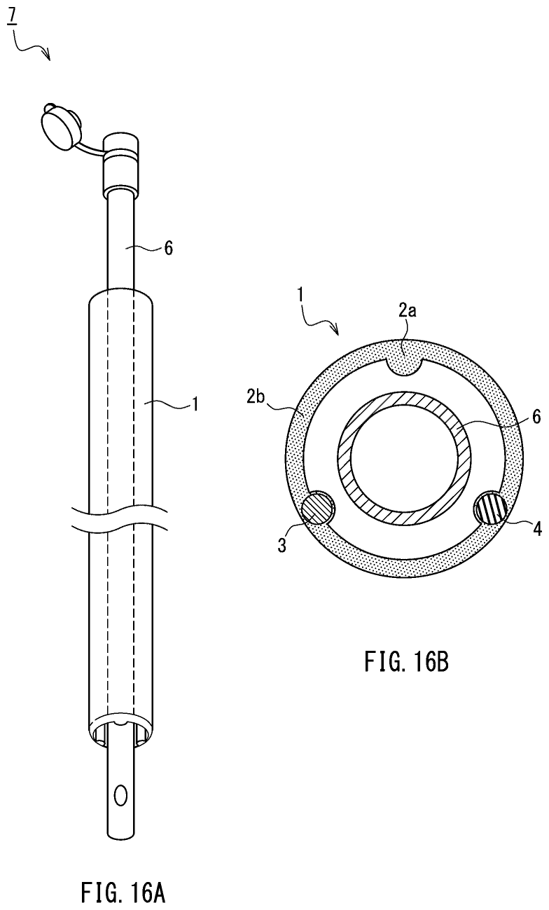

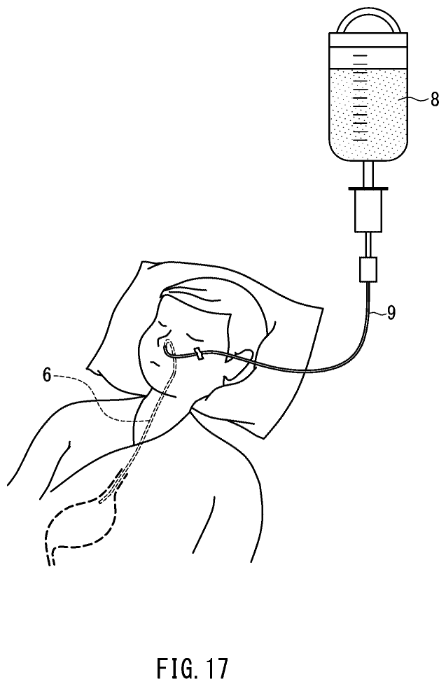

If nutrition taken by the oral ingestion is insufficient, nasal tube feeding is performed after supper, for example. More specifically, the piece of tape is removed from the patient, a feeding catheter 6 is inserted into the inner cavity from the base-side end of the cylindrical component of the indwelled medical-insert guide 1 (FIGS. 16A and 16B). Next, the base end of the feeding catheter 6 is connected with a nutrition set including a nutrition bottle 8 and a circuit 9. The nutrition is supplied to the patient through the feeding catheter 6 by the gravity (see FIG. 17). Before the feeding catheter 6 is inserted into the medical-insert guide 1, the position of the front end of the medical-insert guide 1 may be checked by inserting the camera-including shaft 5 into the medical-insert guide 1.

When the feeding catheter 6 is inserted into the inner cavity of the medical-inset guide 1 indwelled in the body, the front end of the feeding catheter 6 passes, e.g., the nostril, the pharynx, and the esophagus in this order while being in contact with the inner circumferential face of the medical-insert guide 1 and sliding along the inner circumferential face. If the medical-insert guide 1 is not indwelled in the body, the front end of the feeding catheter 6 directly collides with the nostril and the pharynx of the patient. The feeding catheter 6 has a hardness to the degree that maintains the shape of its inner cavity even when the feeding catheter 6 is pressed by the pharynx or the esophagus. Thus, when the front end directly collides, the patient suffers from much pain. If the feeding catheter 6 is inserted into the inner cavity of the medical-insert guide 1 as the guide, the front end of the feeding catheter 6 passes the nostril, the pharynx, and the esophagus while sliding on the inner circumferential face of the cylindrical component of the medical-insert guide 1 without directly colliding with the nostril and the pharynx of the patient. Thus, the above pain of the patient is alleviated. An embodiment where the cylindrical component has a protruding row that protrudes toward the inner cavity and/or toward the outside of the cylindrical component is especially preferable because the protruding row functions as a shock absorbing material.

When the tube nutrition administration ends, the feeding catheter 6 is removed from the medical-insert guide 1. Then, the portion of the medical-insert guide 1 that sticks out of the body from the nose is stuck again to the face of the patient with, e.g., a piece of tape, as illustrated in FIG. 15B. The medical-insert guide 1 continues to be indwelled in the body for approximately two weeks, for example.

Because the medical-insert guide 1 is used together with the feeding catheter they can be marketed as a medical device 7 for nasal tube feeding that includes the medical-insert guide 1 and the feeding catheter 6.

### LIST OF REFERENCE NUMERALS

1, 10, 100 medical-insert guide
2 cylindrical component
22a first layer
22b second layer
2a high-kink-resistance region (thick portion, region A)
2b low-kink-resistance region (thin portion, region B)
21 protruding row
23 a portion of the cylindrical component positioned at the pharynx and the upper esophageal sphincter when the cylindrical component is indwelled in a body
24 insertion-side front end portion
3 optical fiber
4 contrast line
5 camera including shaft
6 feeding catheter
7 medical device for nasal tube feeding

1. A medical-insert guide comprising a cylindrical component,
wherein the cylindrical component is configured to be indwelled in a body from a nose or a mouth to an inside of a gastrointestinal tract, and has an inner cavity into which a medical insert can be removably inserted, and
a portion of the cylindrical component positioned at least at a pharynx and an upper esophageal sphincter when the cylindrical component is indwelled in the body, and an insertion-side front end portion of the cylindrical component each have flexibility that allows a cross-sectional shape of the portion to be deformed into a flat shape by pharynx pressure or esophageal pressure when the cylindrical component is indwelled in the body, or each preliminarily have a flat cross-sectional shape, and each have kink resistance, or
across a whole length of the cylindrical component in its longitudinal direction, the cylindrical component has flexibility that allows its cross-sectional shape to be deformed into a flat shape by pharynx pressure or esophageal pressure when the cylindrical component is indwelled in the body, or preliminarily has a flat cross-sectional shape, and has kink resistance.

2. The medical-insert guide according to claim 1, wherein the cylindrical component has at least one high-kink-resistance region, and the high-kink-resistance region extends across the whole length of the cylindrical component in the longitudinal direction, and is less likely to be subjected to compression deformation in the longitudinal direction than a region of the cylindrical component that is adjacent to the high-kink-resistance region in a circumferential direction of the cylindrical component is likely to be subjected thereto.

3. The medical-insert guide according to claim 2, wherein the cylindrical component has a plurality of the high-kink-resistance regions, and
the high-kink-resistance regions are arranged at predetermined intervals along the circumferential direction of the cylindrical component.

4. The medical-insert guide according to claim 2, wherein the cylindrical component has a thick portion where a cylindrical wall of the cylindrical component is relatively thick, and a thin portion where the cylindrical wall is relatively thin, and the thick portion is the high-kink-resistance region.

5. The medical-insert guide according to claim 4, wherein a ratio of the thickness of the cylindrical component in the thin portion to an outer diameter of the cylindrical component (the thickness in the thin portion/the outer diameter of the cylindrical component) is 0.10 or less.

6. The medical-insert guide according to claim 4, wherein the cylindrical component has the thick portion by having a protruding row that protrudes toward the inner cavity of the cylindrical component.

7. The medical-insert guide according to claim 6, wherein the cylindrical component has a plurality of the protruding rows, and
an imaginary circle that connects tops of the plurality of protruding rows has a diameter of 1.0 mm or more.

8. The medical-insert guide according to claim 4, wherein the cylindrical component is made of at least two types of materials that differ in hardness, and
the hardness of the material of the thick portion is higher than the hardness of the material of the thin portion.

9. The medical-insert guide according to claim 2, wherein a cylindrical wall of the cylindrical component has
a region B and
a region A that is adjacent to the region B in the circumferential direction and is made of a material having a higher hardness than a hardness of a material of the region B, and the region A is the high-kink-resistance region.

10. The medical-insert guide according to claim 1, wherein the cylindrical component has kink resistance performance (length), which is evaluated using a loop method, of 500 mm or less.

11. The medical-insert guide according to claim 1, wherein the insertion-side front end portion of the cylindrical component has a bending strength of 50 g or more, and
the bending strength is measured by pressing an insertion-side front end of the cylindrical component, which is held by its portion that is 3 cm from the insertion-side front end, against a balance, and the bending strength is a weight measured by the balance when the cylindrical component buckles.

12. The medical-insert guide according to claim 1, wherein when the cylindrical component is pressed in a radial direction of the cylindrical component by a pressure of 50 mmHg, the cylindrical component has an outer diameter or thickness of 2.5 mm or less in the pressing direction.

13. The medical-insert guide according to claim 1, wherein the cylindrical component has flexibility to the degree that when the cylindrical component is pressed in a radial direction of the cylindrical component by a pressure of 50 mmHg, the cylindrical component is flattened and the inner cavity of the cylindrical component is eliminated.

14. The medical-insert guide according to claim 1, wherein at least one of an optical fiber or a contrast line is embedded in a cylindrical wall of the cylindrical component and extends across the whole length of the cylindrical component in the longitudinal direction.

15. The medical-insert guide according to claim 1, wherein the cylindrical component has at least two high-kink-resistance regions, and each high-kink-resistance region extends in the longitudinal direction of the cylindrical component, and is less likely to be subjected to compression deformation in the longitudinal direction than a region of the cylindrical component that is adjacent to the high-kink-resistance region in a circumferential direction of the cylindrical component is likely to be subjected thereto,
the cylindrical component includes a flexible hard member, and the flexible hard member is removably inserted in a cylindrical wall of the cylindrical component along the longitudinal direction, is independently present when it is removed from the cylindrical wall, and has kink resistance,
at least one of the at least two high-kink-resistance regions is formed integrally with the adjacent region when the cylindrical component is molded, and
at least one of the at least two high-kink-resistance regions includes the flexible hard member that has been removably inserted in the cylindrical wall along the longitudinal direction.

16. A medical device for nasal tube feeding comprising:
the medical-insert guide according to claim 1; and
a medical insert,
wherein the medical insert is a feeding catheter.
